(19) United States
(12) Patent Application Publication
(10) Pub. No.: US 2005/0065183 A1
(43) Pub. Date: Mar. 24, 2005

(54) Fexofenadine composition and process for preparing

(76) Inventors: Indranil Nandi, Plainsboro, NJ (US); Ashish Anilbhai Patel, Kendall Park, NJ (US); Mohsen Sadatrezaei, Allentown, PA (US); Pablo Davila, East Windsor, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/631,874

(22) Filed: Jul. 31, 2003

(51) Int. Cl. A61K 31/445

(52) U.S. Cl. 514/317; 514/53

(57) ABSTRACT

A pharmaceutical composition comprising fexofenadine or a pharmaceutical acceptable salt thereof, about 10 wt. % to about 70 wt. % of lactose, and about 1 wt. % to about 40 wt. % of a low-substituted hydroxypropyl cellulose, wherein the weight percents are based on the total weight of the pharmaceutical composition. The fexofenadine compositions of the invention exhibit improved bioavailability as expressed as Cₘₐₓ, the maximum amount of active ingredient found in the plasma, or as AUC, the area under the plasma concentration time curve.

### FIELD OF THE INVENTION

The present invention provides a pharmaceutical composition having increased bioavailability which comprises fexofenadine or a pharmaceutical acceptable salt thereof, lactose, and a low-substituted hydroxypropyl cellulose.

### BACKGROUND OF THE INVENTION

It has been established that 4-[4-[4-(hydroxydiphenylmethyl)-1-piperidinyl]-1-hydroxybutyl]-α,α-dimethylbenzeneacetic acid of formula (I) (fexofenadine) is useful as an antihistamine, anti-allergy agent and bronchodilator as disclosed in U.S. Pat. Nos. 3,878,217, 4,254,129 and 4,285,957. Fexofenadine has been shown to have low permeability into central nervous system tissues and weak antimuscarinic activity, causing it to have few systemic side effects.

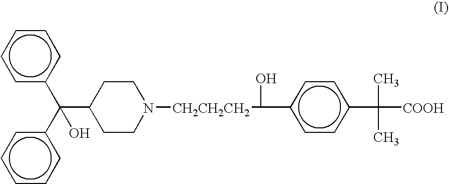

U.S. Pat. No. 4,929,605 describes a pharmaceutical composition in solid unit dosage form containing a therapeutically effective amount of a piperidinoalkanol compound, such as fexofenadine, or a pharmaceutically acceptable salt thereof, a pharmaceutically acceptable nonionic or cationic surfactant in an amount of from about 0.1% to about 6% by weight of the composition, and a pharmaceutically acceptable carbonate salt in an amount of from about 2% to about 50% by weight of the composition.

U.S. Pat. Nos. 5,855,912; 5,932,247; and 6,113,942 describe a pharmaceutical composition in solid unit dosage form containing a piperidinoalkanol compound, microcrystalline cellulose, lactose, pregelatinized starch, gelatin, and croscarmellose sodium.

It would be desirable to develop a fexofenadine composition having improved bioavailability.

### SUMMARY OF THE INVENTION

The invention provides a pharmaceutical composition comprising fexofenadine or a pharmaceutical acceptable salt thereof, about 10 wt. % to about 70 wt. % of lactose, and about 1 wt. % to about 40 wt. % of a low-substituted hydroxypropyl cellulose, wherein the weight percents are based on the total weight of the pharmaceutical composition.

According to another aspect, the invention provides a method of preparing a pharmaceutical composition comprising fexofenadine or a pharmaceutical acceptable salt thereof, about 10 wt. % to about 70 wt. % of lactose, and about 1 wt. % to about 40 wt. % of a low-substituted hydroxypropyl cellulose, wherein the weight percents are based on the total weight of the pharmaceutical composition, said method comprising:
(a) mixing fexofenadine, lactose, low-substituted hydroxypropyl cellulose, and optionally one or more excipients to form a premix;
(b) adding a solvent, preferably water, and optionally a surfactant to the premix formed in Step (a) to form a wet granulation; and
(c) drying the wet granulation to form dried granules;
(d) optionally milling the dried granules; and
(e) mixing at least one excipient with the dried granules to form a pharmaceutical composition.

The fexofenadine compositions of the invention exhibit improved bioavailability as expressed as Cₘₐₓ, the maximum amount of active ingredient found in the plasma, or as AUC, the area under the plasma concentration time curve.

### DESCRIPTION OF THE INVENTION

The pharmaceutical composition of the invention contains fexofenadine, lactose, and a low-substituted hydroxypropyl cellulose. It is noted that fexofenadine may form a salt with various inorganic and organic acids and bases, which salts may be prepared by conventional methods. Suitable inorganic acids are, e.g., hydrochloric, hydrobromic, sulfuric and phosphoric acids. Suitable organic acids include carboxylic acids, such as acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acid; sulfonic acids, such as methanesulfonic, ethanesulfonic and ↑-hydroxyethanesulfonic acid. In addition, “pharmaceutically acceptable salts” include those salts of fexofenidine formed with inorganic and organic bases, such as those of alkali metals, e.g., sodium, potassium and lithium; alkaline earth metals, e.g., calcium and magnesium; light metals of group IIIA, e.g., aluminum; organic amines, e.g., primary, secondary or tertiary amines, such as cyclohexylamine, ethylamine, pyridine, methylaminoethanol and piperazine. As used herein, “fexofenadine” includes pharmaceutical acceptable salts thereof. Preferably, the fexofenadine is fexofenadine hydrochloride.

The amount of fexofenadine or a pharmaceutical acceptable salt thereof in the pharmaceutical compositions is preferably from about 1 wt. % to about 80 wt. %, based on the total weight of the pharmaceutical composition. More preferably, the amount of fexofenadine or a pharmaceutical acceptable salt thereof is from about 5 wt. % to about 50 wt. %, most preferably about 20 wt. % to about 35 wt. %. As indicated above, fexofenadine including pharmaceutical acceptable salts thereof is known and its usefulness as an antihistamine, anti-allergy agent and bronchodilator is also well known. Accordingly, the daily dosages at which said fexofenadine or pharmaceutical acceptable salts thereof are employed as well as typical unit dosages of said fexofenadine or pharmaceutical acceptable salts thereof are well documented in the literature. Preferably, the fexofenadine or a pharmaceutical acceptable salt thereof is present in the pharmaceutical composition in an amount of from about 10 mg to about 200 mg, more preferably 30 to 180 mg.

The lactose is preferably selected from lactose monohydrate, lactose anhydrous, α-lactose, β-lactose. More preferably the lactose is lactose monohydrate. A combination of lactose may also be used. Preferably, the lactose is lactose monohydrate.

The amount of lactose in the pharmaceutical compositions is from about 10 wt. % to about 70 wt. %, preferably, about 25 wt. % to about 65 wt. %, based on the total weight of the pharmaceutical composition. More preferably, the amount of lactose is from about 50 wt. % to about 60 wt. %, based on the total weight of the pharmaceutical composition.

The low-substituted hydroxypropyl cellulose (L-HPC) that is useful in the pharmaceutical compositions is a low-substituted hydroxypropyl ether of cellulose. The L-HPC is available in a number of different grades which have different particle sizes and substitution levels, and which are classified on the basis of their % hydroxypropoxy content. When dried at 105° C. for 1 hour, the L-HPC contains from about 5% to about 16% of hydroxypropoxy groups, preferably from about 10% to about 13% of hydroxypropoxy groups. Suitable grades of L-HPC include the following:
1) LH-11 having a hydroxypropoxy content of 11% and an average particle size of 50 microns;
2) LH-21 having a hydroxypropoxy content of 11% and an average particle size of 40 microns;
3) LH-31 having a hydroxypropoxy content of 11% and an average particle size of 25 microns;
4) LH-22 having a hydroxypropoxy content of 8% and an average particle size of 40 microns;
5) LH-32 having a hydroxypropoxy content of 8% and an average particle size of 25 microns;
6) LH-20 having a hydroxypropoxy content of 13%, and an average particle size of 40 microns; and
7) LH-30 having a hydroxypropoxy content of 13%, and an average particle size of 25 microns.

Preferred L-HPCs are commercially-available from Shin-Etsu Chemical Company under the trade designation L-HPC Grade LH-21 and LH-11.

The amount of the L-HPC in the pharmaceutical compositions is from about 1 wt. % to about 40 wt. %, based on the total weight of the pharmaceutical composition. Preferably, the amount of the L-HPC is from about 2 wt. % to about 25 wt. %, more preferably about 3 wt. % to about 15 wt. %, based on the total weight of the pharmaceutical composition.

In a preferred embodiment, the tablet composition of the invention contains less than 3.5 weight percent, more preferably less than 1 weight percent, based on the weight of the pharmaceutical composition of a binder. Most preferably, the tablet composition does not contain a binder. Examples of binders include starches, e.g., potato starch, wheat starch, corn starch; gums, such as gum tragacanth, acacia gum and gelatin; hydroxypropyl cellulose, hydroxyethyl cellulose, and hydroxypropylmethyl cellulose; and polyvinyl pyrrolidone, e.g., Povidone.

It is within the scope of the invention for the pharmaceutical compositions to include one or more pharmaceutically acceptable excipients. Examples of such excipients are surfactants, enteric-coating agents, diluents, anti-caking agents, amino acids, fibers, solubilizers, disintegrants, fillers, lubricants, emulsifiers, flavorants, solvents, buffers, stabilizers, colorants, dyes, anti-oxidants, anti-adherents, preservatives, electrolytes, glidants and carrier materials. A combination of excipients may also be used. Such excipients are known to those skilled in the art, and thus, only a limited number will be specifically referenced.

Examples of fillers include microcrystalline cellulose, starch, pregelatinized starch, modified starch, dibasic calcium phosphate dihydrate, calcium sulfate trihydrate, calcium sulfate dihydrate, calcium carbonate, dextrose, sucrose, mannitol and sorbitol. A combination of fillers may also be used.

Examples of lubricants include magnesium stearate, calcium stearate, sodium stearate, zinc stearate, talc, propylene glycol, PEG, stearic acid, vegetable oil, sodium benzoate, sodium lauryl sulfate, magnesium lauryl sulfate, mineral oil and polyoxyethylene monostearate. A combination of lubricants may also be used. A preferred lubricant is magnesium stearate.

Examples of disintegrants include:
(i) natural starches, such as maize starch, potato starch and the like, directly compressible starches, e.g., Sta-rx® 1500; modified starches, e.g., carboxymethyl starches and sodium starch glycolate, available as Primojel®, Explotab®, Explosol®; and starch derivatives, such as amylose;
(ii) cross-linked polyvinylpyrrolidones, e.g., crospovidones, such as Polyplasdone® XL and Kollidon® CL;
(iii) alginic acid and sodium alginate;
(iv) methacrylic acid-divinylbenzene co-polymer salts, e.g., Amberlite® IRP-88; and
(v) cross-linked sodium carboxymethylcellulose, available as, e.g., Ac-di-sol®, Primellose®, Pharmacel® XL, Explocel® and Nymcel® ZSX.
   Additional disintegrants also include hydroxypropyl cellulose, hydroxypropylmethyl cellulose, croscarmellose sodium, sodium starch glycolate, polacrillin potassium, polyacrylates, such as Carbopol®, magnesium aluminium silicate and bentonite.

The pharmaceutical compositions of the invention can be prepared by any of the conventionally employed processing techniques such as dry or wet granulation process. Preferably, a wet granulation process is used.

In one embodiment of the invention, the pharmaceutical composition is prepared by a process comprising:
(a) mixing fexofenadine, lactose, and L-HPC, and optionally one or more excipients, to form a premix;
(b) adding a solvent, preferably water, and optionally a surfactant to the premix formed in Step (a) to form a wet granulation;
(c) drying the wet granulation, and optionally milling the dried granules; and
(d) mixing at least one excipient with the granules to form a pharmaceutical composition which is compressed into tablets under conventional conditions as is well known to one of ordinary skill in the art. The compressed tablets can be film coated using standard ingredients and procedures commonly used and well known in the art of pharmaceutical science.

Drying techniques useful for drying granules include spray-drying, fluid bed drying, flash drying, ring drying, micron drying, tray drying, vacuum drying, radio-frequency drying and microwave drying. A preferred drying technique is tray drying. In tray drying, wet granules or wet product is placed on trays which are then placed into a drying oven. The trays are typically made of metal and preferably are lined with plastic. Hot gas or air is circulated over or through the granulation bed.

Milling is a process of reducing larger size granules to smaller size granules in order to achieve proper flow and bulk density in tableting. Types of mills which may be used in the invention include, but are not limited to, fluid energy mill, ball mill or rod mill, hammer mill, cutting mill, and oscillating granulator. More specifically, suitable mills include, Quadro, Fryma, Glatt Quick Sieve, Fluidaire, Fitzpatrick (Fitz mill), BTS mill, and Tornado. A preferred mill is a Quadro Comil which is a conical screen mill and is available from Quadro Inc., Park ridge, N.J. The present inventors have determined that the conical screen mill is very effective for the dry and wet milling of the granules of the invention. In a conical screen mill, granules are fed through an opening in the top of the milling chamber where the granules fall via gravity into a conical screen area with a rotating impellor. The impellor-screen clearance is maintained such that minimal heat is generated and optimum size reduction efficiency is obtained with high throughputs. Variables include screen sizes, impellor designs, and speed.

The pharmaceutical compositions of the invention may be in the form of a capsule, caplet, powder, disc or tablet. In a preferred embodiment, the pharmaceutical compositions are in the form of a tablet.

The following non-limiting examples illustrate further aspects of the invention.

### EXAMPLE 1

Preparation of a fexofenadine tablet composition.

| Ingredient | % | amt/tab |
|---|---|---|
| Fexofenadine HCl | 29.4 | 180.0 |
| Lactose Monohydrate | 56.4 | 345.0 |
| HPC LH-21 | 3.4 | 21.0 |
| Purified Water | None | q.s. |
| HPC LH-21 | 7.8 | 48.0 |
| Magnesium Stearate | 1.0 | 6.0 |
| Core tablet Weight | | 600 mg |
| Opadry ® Clear YS-1-7006 | 2.0 | 12.0 |
| Purified Water | None | q.s. |
| Coated Tablet Weight | 100% | 612 mg |

A pre-mix was prepared using a 800 L Fielder mixer having a plough speed setting #1, chopper speed setting #1 for 5 minutes, which contained fexofenadine HCl, lactose, and hydroxypropyl cellulose. Purified water was added to the pre-mix to form a granulation in the a Fielder. The granulation was dried using a Tray dryer with drying trays at 130° F. The dried granulation was milled using a Quadro Co-mill equipped with a #75 screen. Hydroxypropyl cellulose was added to the milled granulation and mixed using a 566 L Patterson-Kelley Twinshell Blender for 15 minutes. Magnesium Stearate was added through hand screen #20 and mixed using the Twinshell Blender for 3 minutes to form a final mix which was tabletted. The tablets were coated with Opadry® Clear.

### EXAMPLE 2

Preparation of a fexofenadine tablet composition.

| Ingredient | % | amt./tablet |
|---|---|---|
| Fexofenadine HCl | 29.4 | 180.0 |
| Mannitol, USP | 61.3 | 375.0 |
| Purified Water | None | q.s. |
| Silicone Dioxide | 0.5 | 3.0 |
| Polacrilin Potassium | 5.9 | 36.0 |
| Magnesium Stearate | 1.0 | 6.0 |
| Core tablet Weight | | 600 mg |
| Opadry ® Clear YS-1-7006 | 1.9 | 12.0 |
| Purified Water | None | q.s. |
| Coated Tablet Weight | 100% | 612 mg |

A pre-mix was prepared using a 150 L Fielder mixer having a plough speed setting #1, chopper speed setting #1 for 5 minutes, which contained fexofenadine HCl and mannitol. Purified water was added to the pre-mix to form a granulation in the a Fielder mixer. The granulation was dried using a Tray dryer with drying trays at 130° F. The dried granulation was milled using a Fitz mill equipped with a 0.093 inch screen. Silicone dioxide and Polacrilin potassium was added to the milled granulation and mixed using a 142 L Patterson-Kelley Twinshell Blender for 15 minutes. Magnesium Stearate was added through hand screen #20 and mixed using the Twinshell Blender for 3 minutes to form a final mix which was tabletted. The tablets were coated with Opadry® Clear.

### EXAMPLE 3

### Bioavailability Study

The bioavailability was measured in a total of 32 patients who were dosed with the tablets prepared in Example 1 or the tablets prepared in Example 2. Thus, 16 patients received one tablet prepared in Example 1 and 16 patients received one tablet prepared in Example 2. In addition each patent received a reference tablet of Allegra® which is a film coated tablet available from Aventis containing fexofenadine hydrochloride, croscarmellose sodium, magnesium stearate, microcrystalline cellulose, and pregelatinized starch. The Allegra® tablet has a film coating which contains hydroxypropylmethyl cellulose, iron oxide blends, polyethylene glycol, povidone, silicone dioxide, and titanium dioxide. An interval of at least 7 days existed between each patient study. Plasma samples were taken in each patent over a period of 60 hours at time intervals of 0, 0.5, 1, 1.5, 2, 2.5, 3, 4, 5, 6, 7, 8, 10, 12, 18, 24, 30, 36, 48, and 60 hours. The plasma samples were analyzed for the plasma concentration of fexofenadine. The data was expressed as Cₘₐₓ, the maximum amount of fexofenadine found in the plasma, and as AUC, the area under the plasma concentration time curve. The test results are summarized in Table I and Table II.

TABLE I

| AUC | | | Cₘₐₓ | | |
|---|---|---|---|---|---|
| | | Confidence | | | Confidence |
| Allegra ® | Ex. 1 | Interval | | Ex. 1 | Interval |
| (ng/hr/ml) | (ng/hr/ml) | 90% | Allegra ® | (ng/ml) | 90% |
| 4018.96 | 3775.69 | 87.2-101 | 645.24 | 569.94 | 80.7-98.4 |

TABLE II

| AUC | | | Cₘₐₓ | | |
|---|---|---|---|---|---|
| | | Confidence | | | Confidence |
| Allegra ® | Ex. 2 | Interval | | Ex. 2 | Interval |
| (ng/hr/ml) | (ng/hr/ml) | 90% | Allegra ® | (ng/ml) | 90% |
| 3713.47 | 3209.34 | 79.5-101 | 579.42 | 452.71 | 74.5-93.9 |

The results in Tables I and II clearly show that the tablets prepared in Example 1 which were prepared with lactose and low-substituted hydroxypropyl cellulose exhibited a significantly greater bioavailability as determined by AUC and Cₘₐₓ, as compared to the tablets prepared in Example 2 which were prepared with mannitol and polacrilin potassium. In addition, the results in Table I show that the tablets prepared in Example 1 are bioequivalent to the reference product Allegra®.

### EXAMPLE 4

### Evaluation of Drying Method

A first granulation was prepared according to the composition set forth in Example 1. The first granulation was dried using a tray dryer with drying trays at 130° F. The dried granulation was milled using a Quadro Co-mill equipped with a #75 screen. Hydroxypropyl cellulose was added to the milled granulation and mixed using a Patterson-Kelley Twinshell Blender for 15 minutes. Magnesium Stearate was added through hand screen #20 and mixed using the Twinshell Blender for 3 minutes to form a final mix which was tabletted.

A second granulation was prepared according to the composition set forth in Example 1. The second granulation was dried using a fluid bed dryer in which hot air was forced through the granules at a velocity sufficient to partially suspend the granules. The bed of particles is expanded relative to its stationary volume. The particles are continuously being lifted by drag forces from the gas and falling back down under the influence of gravity. The dried granulation was milled using a Quadro Co-mill equipped with a #75 screen. Hydroxypropyl cellulose was added to the milled granulation and mixed using a Patterson-Kelley Twinshell Blender for 15 minutes. Magnesium Stearate was added through hand screen #20 and mixed using the Twinshell Blender for 3 minutes to form a final mix which was tabletted.

The tablets prepared by each of the drying methods were evaluated by dissolving five of each of the tablets prepared by the first granulation and second granulation in a 50/50 mixture by weight of water and acetonitrile. The concentration of fexofenadine was determined by HPLC. The results of the potency assay for tablets prepared by each type of drying method are summarized in Table III.

TABLE III

| Granulation | Drying Method | Potency Assay |
|---|---|---|
| First | Tray Drying | 99.8% |
| Second | Fluid Bed Drying | 94.2% |

The results in Table III show that drying the granulation by a tray dryer resulted in a tablet with a significantly greater amount of fexofenadine as compared to tablets in which the granules were dried using a fluid bed dryer.

### EXAMPLE 5

### Evaluation of Milling Method

A first granulation was prepared according to the composition set forth in Example 1. The first granulation was dried using a tray dryer with drying trays at 130° F. The dried granulation was milled using a low shear Quadro Co-mill equipped with a #75 screen. The granules were fed through an opening in the top of the milling chamber where the granules fell via gravity into a conical screen area with a rotating impellor. The impellor-screen clearance was maintained such that minimal heat is generated and optimum size reduction efficiency was obtained with high throughputs. Hydroxypropyl cellulose was added to the milled granulation and mixed using a Patterson-Kelley Twinshell Blender for 15 minutes. Magnesium Stearate was added through hand screen #20 and mixed using the Twinshell Blender for 3 minutes to form a final mix which was tabletted.

A second granulation was prepared according to the composition set forth in Example 1. The second granulation was dried using a tray dryer with drying trays at 130° F. The dried granulation was milled using a Fitzpatrick mill set at medium speed (approximately 2400 rpm). Hydroxypropyl cellulose was added to the milled granulation and mixed using a Patterson-Kelley Twinshell Blender for 15 minutes. Magnesium Stearate was added through hand screen #20 and mixed using the Twinshell Blender for 3 minutes to form a final mix which was tabletted.

The tablets prepared by each of the milling methods were evaluated by dissolving each of the tablets in water and determining the concentration of fexofenadine. The results of the potency assay for tablets prepared by each type of milling method are summarized in Table IV.

TABLE IV

| Granulation | Milling Method | Potency Assay |
|---|---|---|
| First | Co-Mill | 97.6 |
| Second | Fitzpatrick Mill | 89.5 |

The results in Table IV show that milling the granules using a low shear conical screen mill produced tablets having a significantly greater amount of fexofenadine as compared to tablets in which the granules were milled using a Fitzpatrick mill. While not wishing to be bound by any particular theory, the present inventors believe that high energy milling creates finer granules or particles which also produces dust containing fexofenadine or pharmaceutically acceptable salt thereof, and the generation of dust results in a loss of fexofenadine or pharmaceutically acceptable salt thereof in the pharmaceutical compositions of the invention.

While the invention has been described with particular reference to certain embodiments thereof, it will be understood that changes and modifications may be made by those of ordinary skill within the scope and spirit of the following claims:

1. A pharmaceutical composition comprising fexofenadine or a pharmaceutical acceptable salt thereof, about 10 wt. % to about 70 wt. % of lactose, and about 1 wt. % to about 40 wt. % of a low-substituted hydroxypropyl cellulose, wherein the weight percents are based on the total weight of the pharmaceutical composition.

2. The composition according to claim 1, wherein the salt of fexofenadine is fexofenadine hydrochloride.

3. The composition according to claim 1, wherein the amount of fexofenadine or pharmaceutical acceptable salt thereof is from about 1 wt. % to about 80 wt. %, based on the total weight of the pharmaceutical composition.

4. The composition according to claim 3, wherein the amount of fexofenadine or pharmaceutical acceptable salt thereof is from about 5 wt. % to about 50 wt. %, based on the total weight of the pharmaceutical composition.

5. The composition according to claim 4, wherein the amount of fexofenadine or pharmaceutical acceptable salt thereof is from about 20 wt. % to about 35 wt. %, based on the total weight of the pharmaceutical composition.

6. The composition according to claim 1, wherein the amount of fexofenadine or pharmaceutical acceptable salt thereof is from about 10 mg to about 200 mg.

7. The composition according to claim 6, wherein the amount of fexofenadine or pharmaceutical acceptable salt thereof is from about 30 mg to about 180 mg.

8. The composition according to claim 1, wherein the lactose is selected from the group consisting of lactose monohydrate, lactose anhydrous, α-lactose, β-lactose, and combinations thereof.

9. The composition according to claim 8, wherein the lactose is lactose monohydrate.

10. The composition according to claim 1, wherein the amount of lactose is from about 25 wt. % to about 65 wt. %, based on the total weight of the pharmaceutical composition.

11. The composition according to claim 10, wherein the amount of lactose is from about 50 wt. % to about 60 wt. %, based on the total weight of the pharmaceutical composition.

12. The composition according to claim 1, wherein the low-substituted hydroxypropyl cellulose when dried at 105° C. for 1 hour contains 5-16% of hydroxypropoxy groups.

13. The composition according to claim 12, wherein the low-substituted hydroxypropyl cellulose when dried at 105° C. for 1 hour contains 10-13% of hydroxypropoxy groups.

14. The composition according to claim 13, wherein the low-substituted hydroxypropyl cellulose is selected from the group consisting of: LH-11 having a hydroxypropoxy content of 11% and an average particle size of 50 microns; LH-21 having a hydroxypropoxy content of 11% and an average particle size of 40 microns; LH-31 having a hydroxypropoxy content of 11%, and an average particle size of 25 microns; LH-22 having a hydroxypropoxy content of 8%, and an average particle size of 40 microns; LH-32 having a hydroxypropoxy content of 8%, and an average particle size of 25 microns; LH-20 having a hydroxypropoxy content of 13%, and an average particle size of 40 microns; and LH-30 having a hydroxypropoxy content of 13%, and an average particle size of 25 microns.

15. The composition according to claim 14, wherein the low-substituted hydroxypropyl cellulose is LH-21 or LH-11.

16. The composition according to claim 1, wherein the low-substituted hydroxypropyl cellulose is present in an amount of from about 2 wt. % to about 25 wt. %.

17. The composition according to claim 16, wherein the low-substituted hydroxypropyl cellulose is present in an amount of from about 3 wt. % to about 15 wt. %.

18. A method of preparing a pharmaceutical composition comprising fexofenadine or a pharmaceutical acceptable salt thereof, about 10 wt. % to about 70 wt. % of lactose, and about 1 wt. % to about 40 wt. % of a low-substituted hydroxypropyl cellulose, wherein the weight percents are based on the total weight of the pharmaceutical composition, said method comprising:
(a) mixing fexofenadine, lactose, low-substituted hydroxypropyl cellulose, and optionally one or more excipients to form a premix;
(b) adding a solvent and optionally a surfactant to the premix formed in Step (a) to form a wet granulation; and
(c) drying the wet granulation to form dried granules;
(d) optionally milling the dried granules; and
(e) mixing at least one excipient with the dried granules to form a pharmaceutical composition.

19. A method of preparing a pharmaceutical composition comprising fexofenadine or a pharmaceutical acceptable salt thereof, about 10 wt. % to about 70 wt. % of lactose, and about 1 wt. % to about 40 wt. % of a low-substituted hydroxypropyl cellulose, wherein the weight percents are based on the total weight of the pharmaceutical composition, said method comprising:
(a) mixing fexofenadine, lactose, low-substituted hydroxypropyl cellulose, and optionally one or more excipients to form a premix;
(b) adding a solvent and optionally a surfactant to the premix formed in Step (a) to form a wet granulation; and
(c) drying the wet granulation using a tray dryer to form dried granules;
(d) optionally milling the dried granules using a low shear mill; and
(e) mixing at least one excipient with the dried granules to form a pharmaceutical composition.

20. The method according to claim 19 wherein the low shear mill is a conical screen mill.
